(19) United States
(12) Patent Application Publication
(10) Pub. No.: US 2026/0248479 A1
(43) Pub. Date: Aug. 27, 2026

(54) ULTRASONIC DIAGNOSTIC APPARATUS, MEDICAL IMAGE DIAGNOSTIC APPARATUS, STORAGE MEDIUM, AND METHOD

(71) Applicant: CANON MEDICAL SYSTEMS CORPORATION, Tochigi (JP)

(72) Inventors: Yasunori HONJO, Utsunomiya (JP); Takeshi FUKASAWA, Nasushiobara (JP); Yutaka KOBAYASHI, Nasushiobara (JP); Kazuya AKAKI, Utsunomiya (JP); Atsuko ARAI, Otawara (JP)

(73) Assignee: CANON MEDICAL SYSTEMS CORPORATION, Tochigi (JP)

(21) Appl. No.: 19/550,405

(22) Filed: Feb. 26, 2026

(30) Foreign Application Priority Data
Feb. 27, 2025 (JP) 2025-029899

(51) Int. Cl. A61B 8/06; A61B 8/00; A61B 8/08

(52) U.S. Cl. CPC A61B 8/06; A61B 8/0883; A61B 8/463; A61B 8/488; A61B 8/5246

(57) ABSTRACT

An ultrasonic diagnostic apparatus of an embodiment includes processing circuitry. The processing circuitry acquires a first medical image including tissue information and fluid information of a subject. The processing circuitry specifies a second medical image including tissue information similar to the tissue information included in the first medical image, and including fluid information significantly different from the fluid information included in the first medical image, from a plurality of medical images including the tissue information and the fluid information collected from the subject. The processing circuitry displays a superimposed image in which the fluid information in the first medical image and the fluid information in the second medical image are superimposed in different colors.

### CROSS-REFERENCE TO RELATED APPLICATIONS

This application is based upon and claims the benefit of priority from Japanese Patent Application No. 2025-029899, filed on February 27, 2025; the entire contents of which are incorporated herein by reference.

### FIELD

Embodiments disclosed herein relate generally to an ultrasonic diagnostic apparatus, a medical image diagnostic apparatus, a storage medium, and a method.

### BACKGROUND

Conventionally, a technique for visually observing changes in fluid flow in vivo using a medical image has been known. For example, in the heart valve therapy using catheter intervention, a medical device such as a clip or a prosthetic valve is placed in the heart valve of an object, and the status of blood flow before and after the placement is observed and evaluated using a medical image.

### BRIEF DESCRIPTION OF THE DRAWINGS

FIG. 1 is a block diagram illustrating an example of a configuration of an ultrasonic diagnostic apparatus according to a first embodiment;

FIG. 2 is a diagram for explaining an example of a group of images of an object to be processed according to the first embodiment;

FIG. 3 is a flowchart illustrating the processing procedure of the ultrasonic diagnostic apparatus according to the first embodiment;

FIG. 4 is a diagram illustrating an example of a postoperative image according to the first embodiment;

FIG. 5 is a diagram illustrating an example of a preoperative image according to the first embodiment;

FIG. 6 is a diagram for explaining an example of calculating correction information according to the first embodiment; and

FIG. 7 is a diagram illustrating an example of a superimposed image according to the first embodiment.

### DETAILED DESCRIPTION

An ultrasonic diagnostic apparatus of an embodiment includes processing circuitry. The processing circuitry is configured to acquire a first medical image including tissue information and fluid information of a subject. The processing circuitry is configured to specify a second medical image including tissue information similar to the tissue information included in the first medical image, and including fluid information significantly different from the fluid information included in the first medical image, from a plurality of medical images including the tissue information and the fluid information collected from the subject. The processing circuitry is configured to display a superimposed image in which the fluid information in the first medical image and the fluid information in the second medical image are superimposed in different colors.

Hereinafter, with reference to the accompanying drawings, an embodiment of an ultrasonic diagnostic apparatus, a medical image diagnostic apparatus, a computer program, and a method according to the present application will be described in detail. The ultrasonic diagnostic apparatus, the medical image diagnostic apparatus, the computer program, and the method according to the present application are not limited by the following embodiments.

### First Embodiment

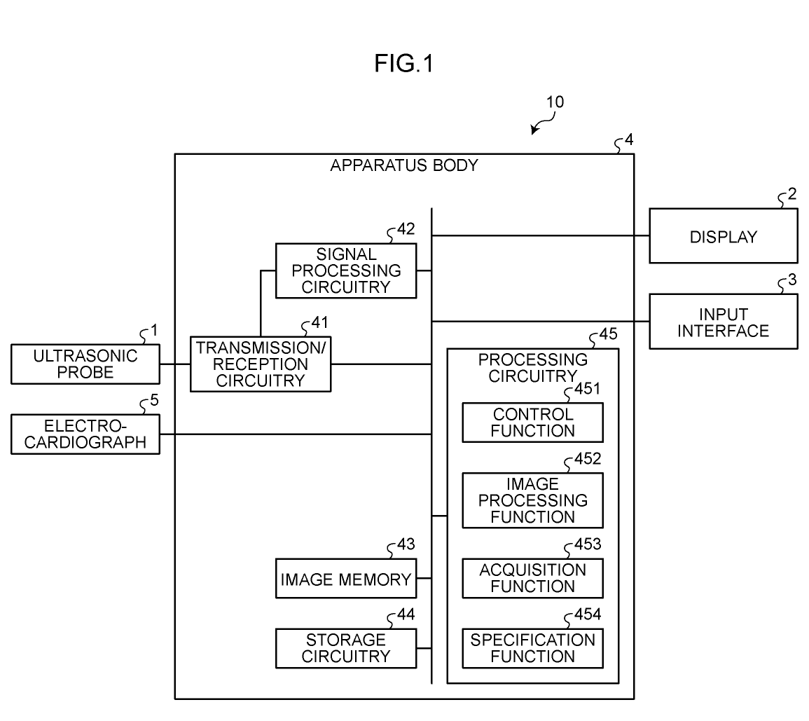

FIG. 1 is a block diagram illustrating an example of a configuration of an ultrasonic diagnostic apparatus 10 according to a first embodiment. As illustrated in FIG. 1, the ultrasonic diagnostic apparatus 10 according to the present embodiment includes an ultrasonic probe 1, a display 2, an input interface 3, and an apparatus body 4. The ultrasonic probe 1, the display 2, and the input interface 3 are communicably connected to the apparatus body 4. Moreover, as illustrated in FIG. 1, the ultrasonic diagnostic apparatus 10 is connected to an electrocardiograph 5.

As a biological signal of a subject P, the electrocardiograph 5 acquires an electrocardiogram (ECG) waveform of the subject. The electrocardiograph 5 transmits the acquired ECG waveform to the apparatus body 4. In the present embodiment, as a means for acquiring information related to the cardiac phase of the heart of a subject, the electrocardiograph 5 is used. However, the embodiment is not limited thereto. For example, the ultrasonic diagnostic apparatus 10 may acquire information related to the cardiac phase of the heart of a subject, by acquiring the time of the sound II (second sound) of the phonocardiogram, or the aortic valve close (AVC) time obtained by measuring the ejection blood flow of the heart using spectral Doppler.

The ultrasonic probe 1 includes a plurality of piezoelectric transducer elements. These piezoelectric transducer elements generate ultrasonic waves on the basis of drive signals supplied from transmission/reception circuitry 41. Moreover, the ultrasonic probe 1 receives reflected waves from the subject, and converts the reflected waves into electrical signals. Furthermore, the ultrasonic probe 1 includes a matching layer provided on the piezoelectric transducer element, a backing material that prevents ultrasonic waves from propagating backward from the piezoelectric transducer element, and the like. The ultrasonic probe 1 is connected to the apparatus body 4 in a detachable manner.

When ultrasonic waves are transmitted from the ultrasonic probe 1 to the subject, the transmitted ultrasonic waves are successively reflected by the discontinuous surface of acoustic impedance in the body tissue of the subject, and are received by the piezoelectric transducer elements of the ultrasonic probe 1 as reflected wave signals. The amplitude of the received reflected wave signals depends on the difference in the acoustic impedance on the discontinuous surface where the ultrasonic waves are reflected. When the transmitted ultrasonic pulse is reflected by the moving blood flow, the surface of the cardiac wall, or the like, due to the Doppler effect, the reflected wave signals depend on the velocity component with respect to the ultrasonic transmission direction of the moving body, and undergo a frequency shift.

The ultrasonic probe 1 may be a one-dimensional ultrasonic probe in which the piezoelectric transducer elements are arranged in a row. Alternatively, the ultrasonic probe 1 may be an ultrasonic probe that mechanically oscillates the piezoelectric transducer elements of a one-dimensional ultrasonic probe, or a two-dimensional ultrasonic probe in which the piezoelectric transducer elements are arranged in a two-dimensional lattice.

The display 2 displays a graphical user interface (GUI) for an operator of the ultrasonic diagnostic apparatus 10 to input various setting requests using the input interface 3, an ultrasonic image generated by the apparatus body 4, and the like. Moreover, the display 2 displays various messages and display information to notify the operator of the processing status and the processing results of the apparatus body 4. Furthermore, the display 2 includes a speaker, and can output sound.

The input interface 3 is operated to perform various settings and the like, and for example, is implemented by a trackball, a switch button, a mouse, a keyboard, a touch pad that performs input operations by touching the operation surface, a touch monitor in which a display screen and a touch pad are integrated, a non-contact input circuit using an optical sensor, a sound input circuit, and the like. The input interface 3 is connected to processing circuitry 45, which will be described below, and converts the input operation received from the operator into an electrical signal, and outputs the converted electrical signal to the processing circuitry 45. In the present specification, the input interface 3 does not necessarily include a physical operation component such as a mouse and a keyboard. For example, an electrical signal processing circuit that receives an electrical signal corresponding to the input operation from an external input device provided separately from the device, and that outputs the electrical signal to the processing circuitry 45 is also an example of the input interface.

The apparatus body 4 is an apparatus that generates an ultrasonic image on the basis of the reflected wave signal received by the ultrasonic probe 1. As illustrated in FIG. 1, the apparatus body 4 includes the transmission/reception circuitry 41, signal processing circuitry 42, an image memory 43, storage circuitry 44, and the processing circuitry 45. The transmission/reception circuitry 41, the signal processing circuitry 42, the image memory 43, the storage circuitry 44, and the processing circuitry 45 are connected to each other in a noticeable manner. In the ultrasonic diagnostic apparatus 10 illustrated in FIG. 1, the processing functions are stored in the storage circuitry 44 in the form of computer-executable programs. The transmission/reception circuitry 41, the signal processing circuitry 42, and the processing circuitry 45 are processors that implement the function corresponding to each computer programs, by reading a computer program from the storage circuitry 44 and executing the read computer program. In other words, each circuitry that has read each computer program has a function corresponding to the read computer program.

The transmission/reception circuitry 41 includes a pulse generator, a transmission delay unit, a pulser, and the like, and supplies a drive signal to the ultrasonic probe 1. The pulse generator repeatedly generates rate pulses for forming transmission ultrasonic waves at a predetermined rate frequency. The transmission delay unit focuses the ultrasonic waves generated by the ultrasonic probe 1 into a beam, and gives the delay time for each of the piezoelectric transducer elements that is necessary to determine the transmission directivity, to each of the rate pulses generated by the pulse generator. The pulser applies a drive signal (drive pulse) to the ultrasonic probe 1 at a timing based on the rate pulse. That is, the transmission delay unit optionally adjusts the transmission direction of the ultrasonic waves transmitted from the surface of the piezoelectric transducer elements, by changing the delay time given to each rate pulse.

To execute a predetermined scan sequence on the basis of the instruction of the processing circuitry 45, which will be described below, the transmission/reception circuitry 41 has a function capable of instantaneously changing the transmission frequency, the transmission drive voltage, and the like. In particular, the transmission drive voltage is changed by a linear-amplifier oscillation circuit capable of instantaneously switching the value, or a mechanism that electrically switches a plurality of power supply units.

Moreover, the transmission/reception circuitry 41 includes a preamplifier, an analog/digital (A/D) converter, a reception delay unit, an adder, and the like, and generates reflected wave data by performing various processes on the reflected wave signals received by the ultrasonic probe 1. The preamplifier amplifies the reflected wave signal for each channel. The A/D converter performs A/D conversion on the amplified reflected wave signal. The reception delay unit gives a delay time necessary for determining the reception directivity. The adder generates reflected wave data by performing an adding process on the reflected wave signal processed by the reception delay unit. By the adding process of the adder, the reflected component from the direction corresponding to the reception directivity of the reflected wave signal is emphasized, and a comprehensive beam for ultrasonic wave transmission and reception is formed by the reception directivity and the transmission directivity.

For example, by performing logarithmic amplification, an envelope detection process, and the like on the reflected wave data received from the transmission/reception circuitry 41, the signal processing circuitry 42 generates data (B-mode data) in which the signal intensity at each sample point is expressed by brightness. The B-mode data generated by the signal processing circuitry 42 is output to the processing circuitry 45.

Moreover, for example, the signal processing circuitry 42 generates data (Doppler data) in which the motion information based on the Doppler effect of a moving body is extracted from the reflected wave data received from the transmission/reception circuitry 41, at each sample point within the scanning region. Specifically, the signal processing circuitry 42 performs frequency analysis on the velocity information from the reflected wave data, extracts echo components of the blood flow, the tissue, and the contrast agent by the Doppler effect, and generates data (Doppler data) obtained by extracting moving body information such as the average velocity, dispersion, and power at multiple points. In this example, the moving body is blood flow, tissue such as a heart wall, or a contrast agent. The motion information (blood flow information) obtained by the signal processing circuitry 42 is sent to the processing circuitry 45, and displayed in color on the display 2 as an average velocity image, a dispersion image, a power image, or an image obtained by combining the average velocity image, the dispersion image, and the power image.

The image memory 43 is a memory that stores image data for display generated by the processing circuitry 45. Moreover, the image memory 43 can store the data generated by the signal processing circuitry 42. For example, the B-mode data and Doppler data stored in the image memory 43 can be called by the operator after diagnosis, and become ultrasonic images for display via the processing circuitry 45.

The storage circuitry 44 stores a control program for performing ultrasonic transmission/reception, image processing, and display processing, and various data such as diagnostic information (for example, patient ID, doctor's observations, and the like), a diagnostic protocol, and various body marks. Moreover, the storage circuitry 44 stores the processing results of the transmission/reception circuitry 41, the signal processing circuitry 42, and the processing circuitry 45. Furthermore, according to the needs, the storage circuitry 44 is used to store image data stored in the image memory 43. Still furthermore, the data stored in the storage circuitry 44 can be transferred to an external apparatus via an interface, which is not illustrated.

The processing circuitry 45 controls the entire process of the ultrasonic diagnostic apparatus 10. Specifically, the processing circuitry 45 controls the processes of the transmission/reception circuitry 41 and the signal processing circuitry 42, on the basis of various setting requests input from the operator via the input interface 3, and various control programs and various data read from the storage circuitry 44. Moreover, the processing circuitry 45 controls the display 2 to display the ultrasonic image for display stored in the image memory 43.

As illustrated in FIG. 1, the processing circuitry 45 performs a control function 451, an image processing function 452, an acquisition function 453, and a specification function 454. In this example, the processing circuitry 45 is an example of processing circuitry.

The control function 451 controls the processes of the transmission/reception circuitry 41 and the signal processing circuitry 42, on the basis of various setting requests input from the operator via the input interface 3, and various control programs and various data read from the storage circuitry 44. For example, the control function 451 performs control according to ultrasonic scanning in various modes. Moreover, the control function 451 controls the display 2 to display an ultrasonic image and various types of information.

The control function 451 displays a superimposed image in which the fluid information in the first medical image and the fluid information in the second medical image are superimposed in different colors. In this example, the first medical image and the second medical image are medical images obtained before and after the change in fluid flow in the subject. For example, the first medical image is an image obtained after a medical device is placed in the body of a subject, and the second medical image is an image obtained before the medical device is placed. Moreover, for example, the fluid in the subject is blood, a contrast agent, or the like. The display control by the control function 451 will be described in detail below.

The image processing function 452 generates an ultrasonic image from the data generated by the signal processing circuitry 42. That is, the image processing function 452 generates an ultrasonic image in which the intensity of the reflected wave from the B-mode data generated by the signal processing circuitry 42 is represented by brightness. Moreover, the image processing function 452 generates an ultrasonic image representing moving body information (blood flow information and tissue moving information) from the Doppler data generated by the signal processing circuitry 42. The ultrasonic image based on Doppler data is velocity image data, dispersion image data, power image data, or image data obtained by combining the velocity image data, the dispersion image data, and the power image data.

In this example, the image processing function 452 generally generates an ultrasonic image for display, by converting (scan converting) a scanning line signal string of ultrasonic scanning into a scanning line signal string in a video format represented by a television, or the like. Specifically, the image processing function 452 generates an ultrasonic image for display, by performing coordinate conversion according to the scanning form of ultrasonic waves by the ultrasonic probe 1. Moreover, in addition to the scan conversion, for example, the image processing function 452 performs various types of image processing, such as image processing (smoothing processing) for regenerating an average value image of luminance, using a plurality of image frames after the scan conversion, image processing using a differential filter (edge enhancement processing) in an image, and the like. Furthermore, the image processing function 452 synthesizes character information, scale, body marks, and the like of various parameters to the ultrasonic image.

That is, the B-mode data and the Doppler data are ultrasonic image data before scan conversion is performed, and the data generated by the image processing function 452 is ultrasonic image data for display after scan conversion is performed. When the signal processing circuitry 42 generates three-dimensional data (three-dimensional B-mode data and three-dimensional Doppler data), the image processing function 452 generates volume data, by performing coordinate conversion according to the scanning form of ultrasonic waves by the ultrasonic probe 1. Then, the image processing function 452 generates two-dimensional image data for display, by performing various rendering processes on the volume data.

The acquisition function 453 acquires the first medical image including the tissue information and the fluid information of the subject. Specifically, the acquisition function 453 acquires the first medical image including the B-mode data and the Doppler data. For example, the acquisition function 453 acquires an ultrasonic image after a medical device is placed in the body of a subject, according to the operation specified by the operator.

From the medical images including the tissue information and the fluid information collected from the subject, the specification function 454 specifies the second medical image including tissue information similar to the tissue information included in the first medical image, and including fluid information significantly different from the fluid information included in the first medical image. For example, from a plurality of ultrasonic images collected before a medical device is placed, the specification function 454 specifies the second medical image in which the tissue information is similar and the fluid information is different from those in the first medical image. The specification process by the specification function 454 will be described in detail below.

An example of the overall configuration of the ultrasonic diagnostic apparatus 10 according to the first embodiment has been described. Under such a configuration, the ultrasonic diagnostic apparatus 10 can easily check the changes in fluid flow in vivo using a medical image. Specifically, by using the ultrasonic images before and after the change in fluid flow in the body of a subject, the ultrasonic diagnostic apparatus 10 automatically specifies the ultrasonic image with a significant degree of change in the fluid flow, and displays the fluid information in the ultrasonic images before and after the change in the fluid flow in different colors in a superimposed manner. Hence, it is possible to easily check the changes in fluid flow in vivo using a medical image.

For example, to perform treatment to prevent regurgitation by placing a medical device in the heart valve using catheter intervention, changes in regurgitation before and after the medical device is placed are evaluated, before the medical device is placed and the treatment is finished. In such treatment, a large number of ultrasonic images are collected and stored during surgery. Hence, it takes time and effort to search the preoperative ultrasonic image to be compared with the postoperative ultrasonic image therefrom.

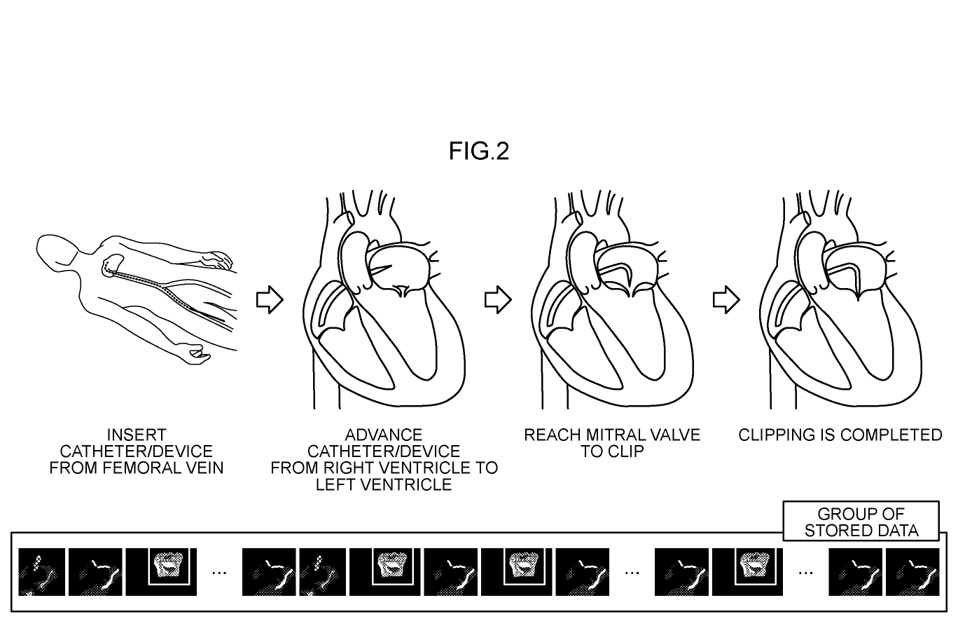

FIG. 2 is a diagram for explaining an example of a group of images of an object to be processed according to the first embodiment. FIG. 2 illustrates a group of medical images collected during the procedure of preventing regurgitation of blood by placing a clip such as the MitraClip (registered trademark) on the mitral valve. In such a procedure, as illustrated in FIG. 2, the mitral valve is clipped, after a catheter and a device are inserted from the femoral vein and advanced to the heart, and a clip is delivered to the mitral valve from the right to left ventricle in the heart. For example, the ultrasonic image is collected to check the general heart function before the catheter and the device are advanced to the heart, or is collected to monitor the tip end when the catheter is advanced to the heart. In addition, the ultrasonic image is collected to check the effects of a clip after the clipping is completed.

For example, before advancing the catheter and the device to the heart (preoperative), to check the status of each portion in the heart such as the aortic valve, mitral valve, left ventricular function, and left atrial appendage, the control function 451 of the ultrasonic diagnostic apparatus 10 collects the ultrasonic images, by performing scanning in various modes such as a "B mode" in which two-dimensional or three-dimensional B-mode data is generated, a "color Doppler mode" in which two-dimensional or three-dimensional color Doppler data is generated, a "PW/CW mode" in which Doppler data indicating a Doppler waveform is generated by a pulsed wave (PW) Doppler method or a continuous wave (CW) Doppler method, and a "bi-plane mode" in which scan data of two orthogonal cross-sections are generated. In this example, the control function 451 collects moving images of a plurality of heartbeats in each mode described above, for example.

Moreover, while the catheter is advanced to the heart, the control function 451 monitors the tip end of the device. Hence, by performing scanning in various modes such as the "B mode", the "color Doppler mode", and the "bi-plane mode", the control function 451 collects the ultrasonic images (moving images of the heartbeats in each mode). After completing the clipping (postoperative), the control function 451 collects the ultrasonic images (moving images of the heartbeats in each mode), by performing scanning in various modes similar to the preoperative.

In this example, the control function 451 collects the ultrasonic images described above, by synchronizing the collection timing by a predetermined trigger. For example, the control function 451 synchronizes the collection timing of image collection in each mode, by start collecting the images in each mode, using a certain time phase (for example, an R wave) in the ECG acquired by the electrocardiograph 5 as a trigger.

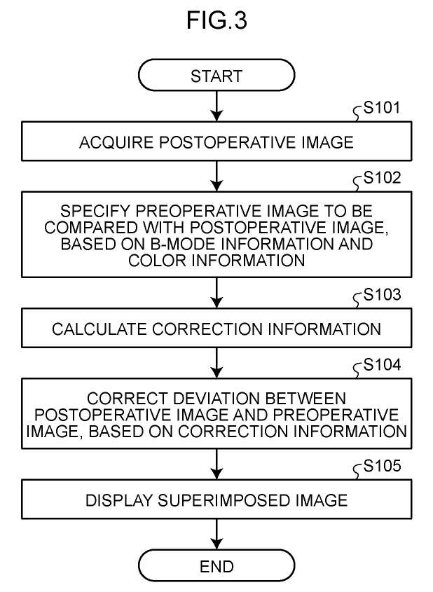

Hereinafter, with reference to FIG. 2, the process performed by the ultrasonic diagnostic apparatus 10 will be described, using as an example the process of specifying the preoperative ultrasonic image to be compared with the postoperative ultrasonic image from a group of stored data, during scanning after the clipping is completed, and displaying the superimposed image. In the following, the process will be described in detail, after describing the processing procedure of the ultrasonic diagnostic apparatus 10. FIG. 3 is a flowchart illustrating the processing procedure of the ultrasonic diagnostic apparatus 10 according to the first embodiment.

For example, as illustrated in FIG. 3, in the present embodiment, the acquisition function 453 acquires the postoperative image (step S101). Specifically, the acquisition function 453 acquires the ultrasonic image (postoperative image) to check the effects of clip placement, from the ultrasonic images collected after the clip is placed by the control of the control function 451. For example, the process at step S101 described above is implemented, when the processing circuitry 45 calls a computer program corresponding to the acquisition function 453 from the storage circuitry 44, and executes the computer program.

Subsequently, on the basis of the B-mode information and the color information of the postoperative image, the specification function 454 specifies the preoperative image to be compared with the postoperative image (step S102), and calculates correction information for correcting the deviation between the specified preoperative image and the postoperative image (step S103). For example, the processes at step S102 and step S103 described above are implemented, when the processing circuitry 45 calls a computer program corresponding to the specification function 454 from the storage circuitry 44, and executes the computer program.

Subsequently, on the basis of the correction information, the control function 451 corrects the deviation between the postoperative image and the preoperative image (step S104), and displays the superimposed image in which the postoperative image and the preoperative image are superimposed, on the display 2 (step S105). For example, the processes at steps S104 and S105 described above are implemented, when the processing circuitry 45 calls a computer program corresponding to the control function 451 from the storage circuitry 44, and executes the computer program.

Hereinafter, the details of each process executed by the ultrasonic diagnostic apparatus 10 will be described.

### Acquisition Process of Postoperative Image

As described at step S101, the acquisition function 453 acquires the preoperative image after a clip is placed on the mitral valve. Specifically, the acquisition function 453 acquires an image included in the moving images synchronously collected by a predetermined trigger, as the postoperative image. In this example, the acquisition function 453 can acquire the preoperative image using various methods. For example, among the ultrasonic images collected after the clip is placed, the acquisition function 453 may acquire the ultrasonic image specified by the operator, as the postoperative image. Alternatively, the acquisition function 453 may acquire the ultrasonic image in the time phase in the ECG set in advance, as the postoperative image. Moreover, among the ultrasonic images collected after the clip is placed, the acquisition function 453 may acquire the ultrasonic image with the maximum fluid information (information indicating regurgitation of blood in the subject), as the postoperative image.

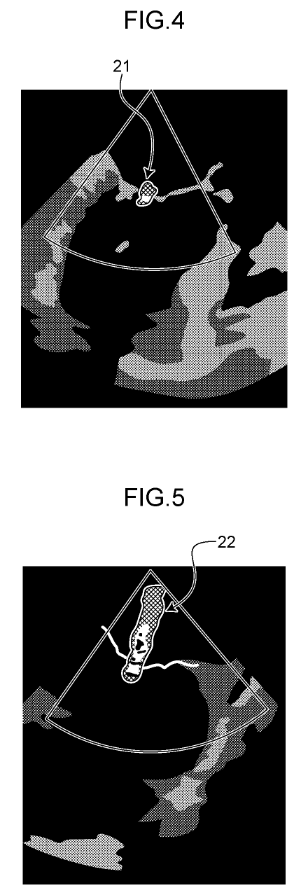

In this example, the acquisition function 453 acquires the postoperative image including the tissue information and the fluid information. For example, between the B-mode data and the color Doppler data synchronously collected on the basis of the time phase in the ECG, the acquisition function 453 acquires the data at the corresponding timing (substantially the same time phase), as the postoperative image. FIG. 4 is a diagram illustrating an example of a postoperative image according to the first embodiment. For example, as illustrated in FIG. 4, the acquisition function 453 acquires an ultrasonic image including the B-mode information indicating the tissue information of the subject, and color information (a colored region 21 in the color Doppler in the drawing) indicating the fluid information of the subject, as the postoperative image.

### Specification Process of Preoperative Image

As described at step S102, the specification function 454 specifies the preoperative image to be compared with the postoperative image, on the basis of the B-mode information and the color information of the postoperative image. Specifically, the specification function 454 acquires an image included in the moving images synchronously collected by a predetermined trigger before surgery, as the preoperative image. In this example, the specification function 454 acquires the preoperative image including the tissue information and the fluid information, as in the case of the postoperative image.

For example, among the moving images collected before surgery, the specification function 454 acquires the B-mode data and the color Doppler data collected at the timing corresponding to the postoperative image (substantially the same time phase), and specifies the B-mode data and the color Doppler data in the preoperative image, using the specification conditions set in advance for each data.

In this example, in the preoperative image to be compared with the postoperative image, it is preferable that the cross section substantially coincides with that of the postoperative image, and the status of regurgitation has changed most. Therefore, the specification function 454 specifies the preoperative image with the similar tissue information and significantly different fluid information. For example, the specification function 454 compares each of the B-mode data collected at the timing corresponding to the postoperative image before surgery, with respect to the B-mode data in the postoperative image, and specifies the most coincident B-mode data as the B-mode data in the preoperative image.

In this case, for example, with the cross-section recognition using artificial intelligence (AI), the specification function 454 calculates the data difference between each of the acquired B-mode data and the B-mode data in the postoperative image, and specifies the B-mode data with the minimum data difference, as the B-mode data in the preoperative image. Alternatively, with the pattern matching using cross-correlation or the like, the specification function 454 may calculate the similarity between each of the acquired B-mode data and the B-mode data in the postoperative image, and specify the B-mode data with the most similarity as the B-mode data in the preoperative image.

Moreover, the specification function 454 compares each of the color Doppler data collected at the timing corresponding to the postoperative image before surgery with the color Doppler data in the postoperative image, and specifies the color Doppler data with the maximum difference in color information, as the color Doppler data in the preoperative image. For example, the specification function 454 specifies the preoperative image (second medical image) with the colored area in the color Doppler the difference of which from the colored area in the color Doppler in the postoperative image (first medical image) becomes maximum, from a plurality of pieces of color Doppler data (a plurality of medical images). That is, among the pieces of color Doppler data collected at the timing corresponding to the postoperative image before surgery, the specification function 454 specifies the color Doppler data in which the color information indicating regurgitation (colored area in the color Doppler) becomes maximum, as the color Doppler data in the preoperative image.

In this example, the specification function 454 specifies the color Doppler data collected under a condition in which the parameter information during scanning is the same or less different, as the color Doppler data in the preoperative image. For example, during scanning, the settings of color system parameters such as image quality may be changed. Therefore, to specify the color Doppler data collected under the similar conditions before and after surgery, the specification function 454 specifies the color Doppler data collected under the condition in which the parameter information during scanning is the same or less different, as the color Doppler data in the preoperative image. That is, the specification function 454 specifies the color Doppler data that is collected under the condition in which the parameter information during scanning is the same or less different, and in which the color information indicating regurgitation becomes maximum, as the color Doppler data in the preoperative image.

As described above, the specification function 454 specifies the preoperative image including tissue information similar to the tissue information of the postoperative image, and fluid information significantly different from the fluid information of the postoperative image. FIG. 5 is a diagram illustrating an example of a preoperative image according to the first embodiment. For example, as illustrated in FIG. 5, the specification function 454 acquires an ultrasonic image including the B-mode information indicating the tissue information of the subject, and the color information indicating the fluid information of the subject (a colored region 22 in the color Doppler in the drawing), as the preoperative image.

### Calculation Process of Correction Information

As described at step S103, the specification function 454 specifies the preoperative image (second medical image), and calculates correction information for correcting the deviation between the postoperative image (first medical image) and the preoperative image. Specifically, the specification function 454 calculates correction information for correcting the deviation at the position of the tissue information and the position at the fluid information (for example, the deviation at the outlet position of regurgitation), between the postoperative image and the preoperative image. In this example, in the treatment described in the present embodiment (placement of a clip on the mitral valve), scanning is performed from an almost fixed position with respect to the heart. Hence, there is often a deviation in the angle of a portion depicted on the ultrasonic image.

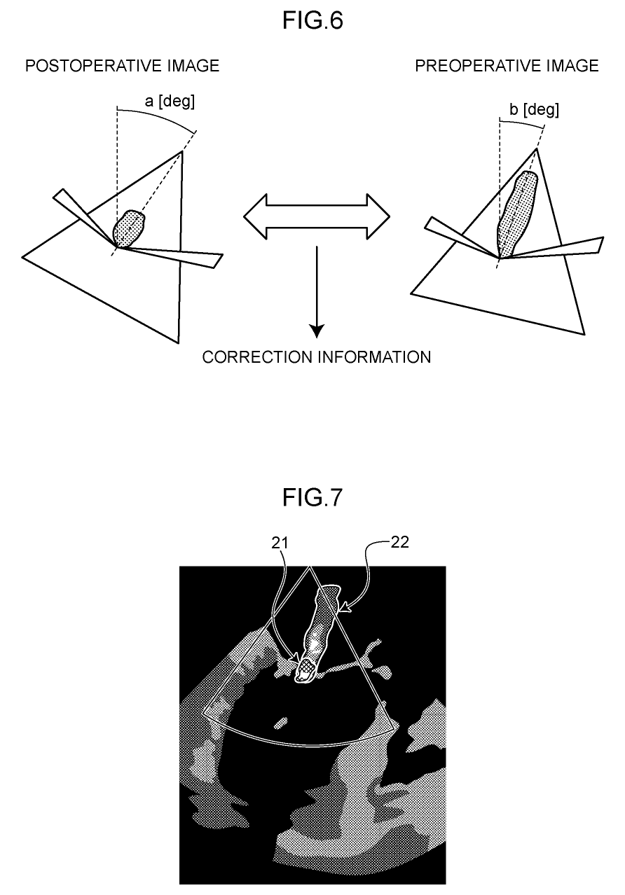

Therefore, the specification function 454 calculates the angle difference between the postoperative image (first medical image) and the preoperative image (second medical image) as correction information. FIG. 6 is a diagram for explaining an example of calculating correction information according to the first embodiment. For example, the specification function 454 calculates the inclination of the mitral valve in the postoperative image (an angle "a [deg]" between any reference and the orientation of the mitral valve), and the inclination of the mitral valve in the postoperative image (an angle "b [deg]" between any reference and the orientation of the mitral valve). Then, the specification function 454 calculates the difference "Φ(b - a)" between the calculated angles, as the correction amount for correcting the inclination between the images.

The correction information is not limited to the correction amount of the inclination described above, and may also be used to calculate the correction amount for correcting the deviation of the upper/lower/left/right positions of the same portion between the images. That is, according to the portion of an object to be compared, the specification function 454 can suitably calculate the correction information for correcting the deviation between the images.

### Correction Process and Display Process

As described at steps S104 and S105, on the basis of the correction information (angle difference), the control function 451 displays a superimposed image in which the deviation between the postoperative image and the preoperative image is corrected. For example, by using the correction amount "Φ(b - a)" for correcting the inclination between the images, the control function 451 corrects the angle difference between the postoperative image and the preoperative image, and displays a superimposed image in which each corrected fluid information (information indicating regurgitation in the postoperative image and information indicating regurgitation in the preoperative image) is superimposed, on the display 2. In this example, the control function 451 can use the B-mode data in the postoperative image or the B-mode data in the preoperative image, as the tissue information.

FIG. 7 is a diagram illustrating an example of a superimposed image according to the first embodiment. For example, as illustrated in FIG. 7, the control function 451 displays a superimposed image in which the colored region 22 in the color Doppler in the preoperative image is superimposed on the postoperative image (B-mode data and the colored region 21 in the color Doppler in the postoperative image), on the display 2. In this example, to identify the colored region 21 in the color Doppler in the postoperative image and the colored region 22 in the color Doppler in the preoperative image, the control function 451 controls to display the colored regions in different colors. For example, by differentiating the numerical value range of RGB to be allocated to the colored region 21 and the numerical value range of RGB to be allocated to the colored region 22, the control function 451 displays the colored region 21 and the colored region 22 in a distinguishable manner.

Moreover, in addition to the superimposed image as described above, the control function 451 can display various superimposed images. For example, the control function 451 displays a display image in which a superimposed image (a superimposed image in which the preoperative fluid information and the postoperative fluid information are superimposed) is superimposed on at least one tissue information between the tissue information included in the postoperative image (first medical image) and the tissue information included in the preoperative image (second medical image). That is, the control function 451 can superimpose the preoperative fluid information and the postoperative fluid information on the various images of the tissue information to display.

For example, the control function 451 displays a display image in which the superimposed image of the colored region 21 in the color Doppler in the postoperative image and the colored region 22 in the color Doppler in the preoperative image illustrated in FIG. 7, is superimposed on the tissue information (B-mode data) included in the preoperative image or the tissue information (B-mode data) included in the postoperative image. Furthermore, for example, the control function 451 can generate a new image from the tissue information (B-mode data) included in the preoperative image and the tissue information (B-mode data) included in the postoperative image, and display a display image in which the superimposed image is superimposed on the generated image. In such a case, for example, a new image may be generated by adding and averaging the B-mode data in the preoperative image and the B-mode data in the postoperative image. Alternatively, a single new image may be generated, by dividing the B-mode data in the preoperative image and the B-mode data in the postoperative image into a plurality of regions, and by selecting and connecting the preoperative image or the postoperative image for each region.

Still furthermore, the control function 451 can display the display image described above in a switchable manner. For example, according to the switching operation by the operator via the input interface 3, the control function 451 can display the display image in which the tissue information superimposed with the fluid information is changed, in a switchable manner.

In addition to displaying the superimposed image as described above, the control function 451 can display the postoperative image and the preoperative image in a parallel manner. That is, the control function 451 can display the postoperative image (the B-mode data and the colored region 21 in the color Doppler in the postoperative image) acquired by the acquisition function 453, and the preoperative image (the B-mode data and the colored region 22 in the color Doppler in the postoperative image) specified by the specification function 454 in a parallel manner, after correcting the inclination using correction information.

Still furthermore, in addition to the superimposed image as described above, the control function 451 can display other information. For example, the control function 451 displays an index value indicating the difference between the fluid information included in the postoperative image (first medical image) and the fluid information included in the preoperative image (second medical image) around the superimposed image. As an example, the control function 451 can display a numerical value indicating the difference between the area of the colored region 21 in the color Doppler and the area of the colored region 22 in the color Doppler around the superimposed image.

The embodiment described above can also be applied to the display image generated from volume data collected in three-dimensions, in addition to the ultrasonic image collected in two-dimensions.

As described above, according to the first embodiment, the acquisition function 453 acquires the first medical image including the tissue information and the fluid information of the subject. From the medical images including the tissue information and the fluid information collected from the subject, the specification function 454 specifies the second medical image including tissue information similar to the tissue information included in the first medical image, and including fluid information significantly different from the fluid information included in the first medical image. The control function 451 displays a superimposed image in which the fluid information in the first medical image and the fluid information in the second medical image are superimposed in different colors. Thus, the ultrasonic diagnostic apparatus 10 according to the first embodiment can automatically specify the medical image that can accurately identify how the fluid information has changed between the first medical image (postoperative image) and the second medical image (preoperative image), and can display the difference between these medical images in a visually recognizable manner. As a result, the ultrasonic diagnostic apparatus 10 can easily check the changes in fluid flow in vivo using a medical image.

For example, by creating the superimposed image as described above, in addition to arranging the medical images to be compared in a parallel manner on the postoperative report, it is possible to create a convincing report that can visually store the reduction in regurgitation due to the procedure in a single image.

Moreover, according to the first embodiment, the first medical image is an image obtained after a medical device is placed in the body of a subject, and the second medical image is an image obtained before the medical device is placed. Thus, the ultrasonic diagnostic apparatus 10 according to the first embodiment can easily check the changes in fluid flow in vivo, in the medical images collected before and after the procedure of placing the medical device.

Furthermore, according to the first embodiment, the fluid information is information indicating regurgitation of blood in the subject. Thus, the ultrasonic diagnostic apparatus 10 according to the first embodiment can easily check the effects of the procedure for regurgitation.

Still furthermore, according to the first embodiment, the first medical image and the second medical image are images each included in the moving images synchronously collected by a predetermined trigger. Thus, the ultrasonic diagnostic apparatus 10 according to the first embodiment can make a comparison between the images collected at the same timing. Hence, it is possible to make an appropriate comparison.

Still furthermore, according to the first embodiment, the predetermined trigger is a certain time phase in the ECG waveform. Thus, the ultrasonic diagnostic apparatus 10 according to the first embodiment can easily synchronize the collection timing.

Still furthermore, according to the first embodiment, the specification function 454 specifies the second medical image with the colored area in the color Doppler the difference of which from the colored area in the color Doppler in the first medical image becomes maximum, from the medical images. Thus, for the fluid information, the ultrasonic diagnostic apparatus 10 according to the first embodiment can specify an appropriate object to be compared.

Still furthermore, according to the first embodiment, the control function 451 displays an index value indicating the difference between the fluid information included in the first medical image and the fluid information included in the second medical image around the superimposed image. Thus, the ultrasonic diagnostic apparatus 10 according to the first embodiment can display information on the comparison results, and can easily check the changes in fluid flow in vivo.

Still furthermore, according to the first embodiment, the first medical image and the second medical image are two-dimensional images or three-dimensional images. Thus, the ultrasonic diagnostic apparatus 10 according to the first embodiment can apply the process described above on the ultrasonic images collected by various procedures.

Still furthermore, according to the first embodiment, the specification function 454 specifies the second medical image, and calculates correction information for correcting the deviation between the first medical image and the second medical image. Thus, the ultrasonic diagnostic apparatus 10 according to the first embodiment can easily acquire the correction information.

Still furthermore, according to the first embodiment, the correction information is the angle difference between the first medical image and the second medical image, and the control function 451 displays a superimposed image in which the deviation between the first medical image and the second medical image is corrected on the basis of the angle difference. Thus, the ultrasonic diagnostic apparatus 10 according to the first embodiment can easily correct the deviation between the first medical image and the second medical image.

### Other Embodiments

In the embodiment described above, the preoperative image and the postoperative image are compared in catheter intervention treatment for mitral valve regurgitation. However, the embodiment is not limited thereto, and the preoperative image and the postoperative image may also be compared in radiofrequency ablation (RFA) treatment.

Moreover, in the embodiment described above, the first medical image is the postoperative image, and the second medical image is the preoperative image. However, the embodiment is not limited thereto, and the first medical image may be the preoperative image and the second medical image may be the postoperative image.

Furthermore, in the embodiment described above, the preoperative image and the postoperative image are compared. However, the embodiment is not limited thereto, and the medical images collected before and after the exercise load is applied may also be compared. That is, to compare the status of blood flow before the exercise load is applied with the status of blood flow after the exercise load is applied, the process described above may be executed to display the blood flow information in a superimposed manner.

Still furthermore, in the embodiment described above, the fluid information is blood flow. However, the embodiment is not limited thereto, and for example, the fluid information may also be a contrast agent.

Still furthermore, in the embodiment described above, a still image is superimposed. However, the embodiment is not limited thereto, and a moving image may be also displayed in a superimposed manner. That is, of at least one of the fluid information in the first medical image and the fluid information in the second medical image superimposed on the superimposed image, the control function 451 can display the corresponding fluid information in the moving images on the superimposed image as a moving image.

For example, to display the postoperative colored region 21 in the color Doppler illustrated in FIG. 7 as a moving image, the control function 451 acquires the postoperative color Doppler data corresponding to each of the time phases in the ECG including the colored region 21, and continuously displays the color Doppler data acquired in each of the time phases, in the order of time phases on the B-mode data illustrated in FIG. 7. Consequently, it is possible to display the moving image indicating the postoperative status of regurgitation of blood.

Moreover, for example, to display the colored region 22 of the preoperative color Doppler illustrated in FIG. 7 as a moving image, the control function 451 acquires the preoperative color Doppler data corresponding to each of the time phases in the ECG including the colored region 22, and continuously displays the color Doppler data acquired in each of the time phases on the B-mode data illustrated in FIG. 7 in a superimposed manner, in the order of time phases. Consequently, it is possible to display a moving image indicating the preoperative status of regurgitation of blood.

Furthermore, for example, to display each of the colored region 21 and the colored region 22 as a moving image, the control function 451 continuously displays the postoperative color Doppler data in each of the time phases and the preoperative color Doppler data in each of the time phases acquired as described above, on the B-mode data illustrated in FIG. 7 by adjusting the time phase, in the order of time phases. Consequently, it is possible to display the moving image indicating the preoperative status of regurgitation of blood and the postoperative status of regurgitation of blood.

In this example, to display each of the preoperative fluid information and the postoperative fluid information as a moving image, the moving image to be displayed may be determined using the difference between the pieces of fluid information of the entire moving images to be compared and displayed. In such a case, to display each of the fluid information in the first medical image and the fluid information in the second medical image as a moving image on the superimposed image, the specification unit integrates the difference between the colored areas of the color Doppler at corresponding time phases, and specifies the moving image in which the integrated difference becomes maximum.

For example, the specification function 454 calculates each difference between the colored areas of the color Doppler, by comparing the color Doppler data in each of the time phases collected after surgery (moving image including the postoperative color Doppler data acquired by the acquisition function 453) and the color Doppler data in each of the time phases collected before surgery (moving image of color Doppler) at corresponding time phases, and calculates the integrated difference obtained by integrating the calculated difference. For the moving image corresponding to each heartbeat in the moving images of the heartbeats collected before surgery, the specification function 454 calculates the integrated difference described above, and specifies the moving image in which the integrated difference becomes maximum, as the moving image to be compared and displayed.

As described above, by displaying the fluid information as a moving image, it is possible to easily identify the overall changes in the fluid information. The tissue information may also be displayed as a moving image, in addition to displaying the fluid information as a moving image as described above. That is, the control function 451 can display at least one of the B-mode data in each of the time phases collected after surgery and the B-mode data in each of the time phases collected after surgery on the superimposed image as a moving image.

Moreover, in the embodiment described above, the ultrasonic diagnostic apparatus executes the method according to the present application. However, the embodiment is not limited thereto, and the method according to the present application may also be executed by another medical image diagnostic apparatus such as an X-ray computed tomography (CT) apparatus and a magnetic resonance imaging (MRI) apparatus, or by a medical image processing apparatus such as a workstation.

For example, the term "processor" used in the above description refers to a central processing unit (CPU), a graphics processing unit (GPU), or a circuit such as an application specific integrated circuit (ASIC) and a programmable logic device (for example, a simple programmable logic device (SPLD), a complex programmable logic device (CPLD), and a field programmable gate array (FPGA)). The processor implements the function by reading and executing the computer program stored in the memory. Instead of storing a computer program in the memory, the computer program may be directly incorporated into the circuit of the processor. In this case, the processor implements the function by reading and executing the computer program incorporated in the circuit. Each processor in the present embodiment is not limited to being configured as a single circuit for each processor, but may also be configured as a single processor by combining a plurality of independent circuits to implement the function.

Each component of each apparatus illustrated in the description of the above embodiments is functionally conceptual, and need not be physically configured as illustrated in the drawings. That is, the specific mode of dispersion and integration of each apparatus is not limited to the ones illustrated in the drawings, and all or a part thereof can be functionally or physically dispersed or integrated in an optional unit, depending on various kinds of loads, the status of use, and the like. Moreover, all or any part of the processing functions performed by the apparatuses may be implemented by the CPU or a computer program analyzed and executed by the CPU, or can be implemented as hardware using wired logic.

Furthermore, the methods described in the embodiments described above can be implemented by executing a computer program prepared in advance on a computer such as a personal computer or workstation. This computer program can be distributed via a network such as the Internet. Moreover, this computer program may be recorded on a computer-readable non-transitory recording medium such as a hard disk, a flexible disk (FD), a CD-ROM, an MO, a DVD, and a flash memory such as a USB memory and a SD card memory, and executed by being read from the non-transitory recording medium by a computer.

As described above, according to the embodiments, it is possible to easily check the changes in fluid flow in vivo using a medical image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus, comprising:
processing circuitry configured to
acquire a first medical image including tissue information and fluid information of a subject;
specify a second medical image including tissue information similar to the tissue information included in the first medical image, and including fluid information significantly different from the fluid information included in the first medical image, from a plurality of medical images including the tissue information and the fluid information collected from the subject; and
display a superimposed image in which the fluid information in the first medical image and the fluid information in the second medical image are superimposed in different colors.

2. The ultrasonic diagnostic apparatus according to claim 1, wherein the first medical image is an image obtained after a medical device is placed in a body of the subject, and the second medical image is an image obtained before the medical device is placed.

3. The ultrasonic diagnostic apparatus according to claim 2, wherein the fluid information is information indicating regurgitation of blood in the subject.

4. The ultrasonic diagnostic apparatus according to claim 1, wherein the first medical image and the second medical image are images each included in a plurality of moving images synchronously collected by a predetermined trigger.

5. The ultrasonic diagnostic apparatus according to claim 4, wherein the processing circuitry is configured to display, as a moving image within the superimposed image, the fluid information corresponding to at least one of the fluid information in the first medical image and the fluid information in the second medical image superimposed on the superimposed image.

6. The ultrasonic diagnostic apparatus according to claim 4, wherein the predetermined trigger is a certain time phase in an ECG waveform.

7. The ultrasonic diagnostic apparatus according to claim 1, wherein the processing circuitry is configured to specify a second medical image with a colored area in a color Doppler difference of which from a colored area in a color Doppler in the first medical image becomes maximum, from the plurality of medical images.

8. The ultrasonic diagnostic apparatus according to claim 5, wherein, to display each of the fluid information in the first medical image and the fluid information in the second medical image as a moving image on the superimposed image, the processing circuitry is configured to integrate difference between colored areas of color Doppler at corresponding time phases, and specify a moving image with maximum integrated difference.

9. The ultrasonic diagnostic apparatus according to claim 1, wherein the processing circuitry is configured to display an index value indicating difference between the fluid information included in the first medical image and the fluid information included in the second medical image around the superimposed image.

10. The ultrasonic diagnostic apparatus according to claim 1, wherein the first medical image and the second medical image are two-dimensional images or three-dimensional images.

11. The ultrasonic diagnostic apparatus according to claim 1, wherein the processing circuitry is configured to display a display image in which the superimposed image is superimposed on at least one tissue information between the tissue information included in the first medical image and the tissue information included in the second medical image.

12. The ultrasonic diagnostic apparatus according to claim 1, wherein the processing circuitry is configured to specify the second medical image, and calculate correction information for correcting deviation between the first medical image and the second medical image.

13. The ultrasonic diagnostic apparatus according to claim 12, wherein
the correction information is an angle difference between the first medical image and the second medical image, and
the processing circuitry is configured to display a superimposed image in which the deviation between the first medical image and the second medical image is corrected, based on the angle difference.

14. A medical image diagnostic apparatus, comprising:
processing circuitry configured to
acquire a first medical image including tissue information and fluid information of a subject;
specify a second medical image including tissue information similar to the tissue information included in the first medical image, and including fluid information significantly different from the fluid information included in the first medical image, from a plurality of medical images including the tissue information and the fluid information collected from the subject; and
display a superimposed image in which the fluid information in the first medical image and the fluid information in the second medical image are superimposed in different colors.

15. A non-transitory computer readable medium comprising instructions that cause a computer to execute:
acquiring a first medical image including tissue information and fluid information of a subject;
specifying a second medical image including tissue information similar to the tissue information included in the first medical image, and including fluid information significantly different from the fluid information included in the first medical image, from a plurality of medical images including the tissue information and the fluid information collected from the subject; and
displaying a superimposed image in which the fluid information in the first medical image and the fluid information in the second medical image are superimposed in different colors.

16. A method, comprising:
acquiring a first medical image including tissue information and fluid information of a subject;
specifying a second medical image including tissue information similar to the tissue information included in the first medical image, and including fluid information significantly different from the fluid information included in the first medical image, from a plurality of medical images including the tissue information and the fluid information collected from the subject; and
displaying a superimposed image in which the fluid information in the first medical image and the fluid information in the second medical image are superimposed in different colors.
